(19) United States
(12) Patent Application Publication
(10) Pub. No.: US 2025/0380876 A1
(43) Pub. Date: Dec. 18, 2025

(54) PORTABLE DEVICE FOR DETECTING A CONSTRICTION, AND SYSTEM EQUIPPED WITH SAME

(71) Applicant: FRESENIUS MEDICAL CARE DEUTSCHLAND GMBH, Bad Homburg (DE)

(72) Inventors: Hendrik KUPPER, Bad Homburg (DE); Gerome FISCHER, Bad Homburg (DE); Alicia HAUKE, Bad Homburg (DE)

(73) Assignee: FRESENIUS MEDICAL CARE DEUTSCHLAND GMBH, Bad Homburg (DE)

(21) Appl. No.: 19/112,282

(22) PCT Filed: Sep. 20, 2023
(86) PCT No.: PCT/EP2023/075921

§ 371 Date: Mar. 17, 2025

(30) Foreign Application Priority Data
Sep. 20, 2022 (DE) 10 2022 209 907.9

(51) Int. Cl. A61B 5/02; A61B 5/00; A61B 5/026

(52) U.S. Cl. CPC A61B 5/02007; A61B 5/026; A61B 5/6804; A61B 2560/0431; A61B 2562/0247

(57) ABSTRACT

The invention relates to a portable device (1) for the detection of a constriction stenosis in a lumen, wherein the wearable device has a membrane-like substrate (S) which, in use, is arranged on the outside of the lumen, wherein at least two sound converters (M1, M2) are arranged on the membrane-like substrate (S), wherein each sound converter (M1,M2) has at least one stack of layers, wherein the stack of layers comprises at least one stack of layers, wherein the stack of layers comprises at least one partially covering sequence of a first electrically conductive layer, a PVDF layer and a second electrically conductive layer, wherein the PVDF layer is polarised electrically perpendicularly to the layer structure and has a piezo effect, wherein on the side facing away from the substrate furthermore a biocompatible cover layer (DS) is provided, wherein the first electrically conductive layer and the second electrically conductive layer are able to be connected to an evaluation unit (AE).

### BACKGROUND

In many areas, the detection of constrictions of lumens is relevant. Particularly, in many fields of medicine, therapies exist in which a stenosis may occur.

In the following, reference is made further to haemodialysis without, however, hereby restricting the invention to this application alone.

Haemodialysis is a treatment which, in the case of a renal dysfunction, whether acute or chronic, permits the function of the kidney to be supplemented. Here, typically (arterial) blood is processed in an external blood circulation and is subsequently delivered again to the (venous) blood circulation.

Typically for this a vascular access at an upper extremity is used. Widely used vascular accesses are so-called arteriovenous fistulas or central venous catheters.

This vascular access is intended to enable high throughflow rates, in order to enable an effective and fast treatment.

Faulty vascular accesses can be demonstrated for over 20% of the hospitalizations of dialysis patients—not all of which end favourably.

The most frequent source of error at the vascular access or respectively in its vicinity are stenoses, i.e. a vascular constriction, or thromboses, i.e. a vascular occlusion.

Therefore, the monitoring of the functionality of a vascular access during a dialysis is a suitable provision, in order to reduce the number of emergencies or the mortality overall.

Currently, vascular accesses are monitored predominantly by monitoring with a stethoscope. However, such a monitoring mostly only occurs once, so that a stenosis occurring in the interim may not be detected. In addition, such a monitoring requires a trained ear. This means that it may occur that one person detects an (impending) stenosis, whereas another person does not detect an (impending) stenosis.

If an impending stenosis is detected promptly, this can be verified through corresponding imaging. Typical provisions for imaging are e.g. (colour) doppler ultrasound examinations. These allow e.g. false positive suspected diagnoses to be ruled out.

However, constrictions can also occur elsewhere. For example, it is known in medicine that when treating patients, constrictions can form in lumens leading to or from the patient, which can also prevent further flow. This occurs, for example, during extracorporeal blood treatments—especially with blood sets. There can be various reasons for the constriction. For example, a tube may be kinked or jammed or otherwise under strain—e.g. the patient is lying on a tube. It would also be advantageous to provide a simple, low-cost option for monitoring such devices.

Approaches hitherto for a continuous monitoring provide the monitoring/evaluation of the (venous) pump pressure or flow differences between the inflow and the outflow, total flow amount, etc.

By way of example, reference is to be made to the German patent application DE 10 2013 213 390 A1, from which such a device is known.

A disadvantage in the approaches hitherto is that they require a complex configuration of the dialysis device. The reason for this is e.g. in that equipment is not provided only for one patient, but rather after a completed disinfection, it is to be available again for other patients. Therefore, high demands are placed on the disinfectability. This frequently means a very complex construction and/or a complex disinfection.

In addition, the availability is restricted only to newly delivered equipment.

Recently, sensors based on polyvinylidene fluorides are proposed, the audio signals of which are evaluated directly, e.g. as a phonoangiogram.

However, these sensors have, inter alia, the disadvantage that polyvinylidene fluorides must be conditioned by means of high voltage in order to be able to achieve the desired effect.

In the approaches hitherto, which provide a connection by means of layers comprising silver, with an exertion of pressure short circuits frequently arise, which are an obstacle to the functionality.

Therefore the production is costly and the use of such systems for the production of sound converters was hitherto not pursued consistently.

### OBJECT

Against this background, it is an object of the invention to provide an economical device which makes it possible to detect an (impending) stenosis reliably and securely.

### BRIEF PRESENTATION OF THE INVENTION

The problem is solved by a portable device for the detection of a constriction in a lumen, wherein the portable device has a membrane-like substrate which, upon use, is arranged on the outside of the lumen, wherein at least two sound converters are arranged on the membrane-like substrate, wherein each sound converter comprises at least one stack of layers, wherein the stack of layers comprises at least one partially covering sequence of a first electrically conductive layer, a PVDF layer and a second electrically conductive layer, wherein the PVDF layer is electrically polarised perpendicularly to the layer structure and has a piezo effect, wherein on the side facing away from the substrate furthermore a biocompatible cover layer is provided, wherein the first electrically conductive layer and the second electrically conductive layer are able to be connected to an evaluation unit.

Such devices can be produced economically and permit a reliable and secure detection of (impending) stenoses.

According to a further development of the invention, the first electrically conductive layer and/or the second electrically conductive layer comprises an electrically conductive plastic, in particular an electrically conductive polymer, and preferably PEDOT.PSS.

According to a further development of the invention, the membrane-like substrate has a carrier membrane comprising PET, PU, PEN, PI or PC. Thus, on the basis of different material systems, a device can be provided which can also be combined with other arrangements, without this being ruled out through the choice of a particular material system.

In a further further development of the invention, the at least two sound converters are pressure sensors or microphones or structure-borne sound converters.

This means that the invention permits diverse variation possibilities.

According to a further development of the invention, the stack of layers comprises several sequences of a first electrically conductive layer, a PVDF layer and a second electrically conductive layer.

Hereby, the sensitivity can be improved.

In one more further further development of the invention, the stack of layers comprises several sequences of a first electrically conductive layer, a PVDF layer and a second electrically conductive layer, wherein the second electrically conductive layer of a first sequence also forms at the same time the first electrically conductive layer of a second sequence.

Thus with simultaneous increasing of the sensitivity, the stack size can be minimised.

According to a still further further development of the invention, the cover layer comprises PDMS or PET. In particular, the cover layer can provide a mechanical impedance matching between the skin and an electrically conductive layer situated under the cover layer. Alternatively and/or additionally, the cover layer can also serve as contact protection and/or corrosion protection.

According to a further further development of the invention, at least the stack of layers is embedded in a non-conducting material. In particular the non-conducting material is PET or PU.

Hereby, a contact protection and/or corrosion protection can also be provided as also an additional mechanical stability for the layer structure.

In a still further further development of the invention, the connections of the first electrically conductive layer and/or of the second electrically conductive layer with the evaluation unit have a layer comprising silver.

Hereby, a secure, low-impedance connection can be provided.

According to a still further further development of the invention, at least one further electrically conducting layer is arranged laterally adjacent to a stack.

By means of such further electrical layers, a shielding against interfering irradiations can be provided.

According to still another embodiment of the invention, the portable device is configured to be worn on the body of a human or animal body, wherein, in use, the portable device is arranged on the skin of the human or animal body to detect a stenosis in a blood vessel of the human or animal body.

The problem is solved furthermore by a system for the detection of constrictions, having a portable device according to the invention and an evaluation unit, wherein the evaluation unit is connected to the at least two sound converters, wherein the signals from each two sound converters are repeatedly evaluated differentially, wherein on the occurrence of a significant phase shift and/or of a significant signal change at only one sound converter between a first evaluation and a second evaluation, following the first chronologically, a stenosis is detected. In particular, in a further development of the invention, provision can be made that a significant phase shift has a phase shift of approximately 90°.

Such systems can be produced economically and permit a reliable and secure detection of (impending) stenoses. In particular, here, the portable device can be configured as a disposable article, whereas the evaluation unit can be re-used.

Further advantageous configurations are the subject of the respectively dependent claims, the figures and the description.

### BRIEF PRESENTATION OF THE FIGURES

The invention is explained more closely in the following by means of a drawing and example embodiments. The drawing is a schematic representation and is not true to scale. The drawing does not restrict the invention in any way.

There are shown:

FIG. 1 a schematic representation of an embodiment of the invention,

FIG. 2 a schematic representation of a further embodiment of the invention,

FIG. 3 a schematic representation of an aspect of embodiments of the invention, and

FIG. 4 a schematic representation of further aspects of embodiments of the invention.

### DETAILED PRESENTATION OF THE INVENTION

The invention will be presented in the following in more detail with reference to the figures. It is to be noted here that different aspects are described which can be used respectively individually or in combination. This means that any aspect can be used with different embodiments of the invention in so far as not explicitly presented as a pure alternative.

Furthermore, in the following, for the sake of simplicity generally always reference is made to only one entity. In so far as not explicitly mentioned, however, the invention can also have respectively several of the entities concerned. In this respect, the use of the words “a”, “an”, “of a” is to be understood only as an indication that in a single embodiment at least one entity is used.

In so far as methods are described in the following, the individual steps of a method are able to be arranged and/or combined in any desired sequence, in so far as something different does not result explicitly through the context. Furthermore, the methods—in so far as not expressly characterized otherwise—are able to be combined with one another.

Data with numerical values are generally not to be understood as exact values, but rather also contain a tolerance from +/−1% up to +/−10%.

In so far as in this application standards, specifications or suchlike are named, at least always reference is made to the standards, specifications or suchlike which are able to be used on the application date. This means that if a standard/specification etc. is updated or replaced by a successor, the invention is also able to be used subsequently.

Various embodiments are illustrated in the figures.

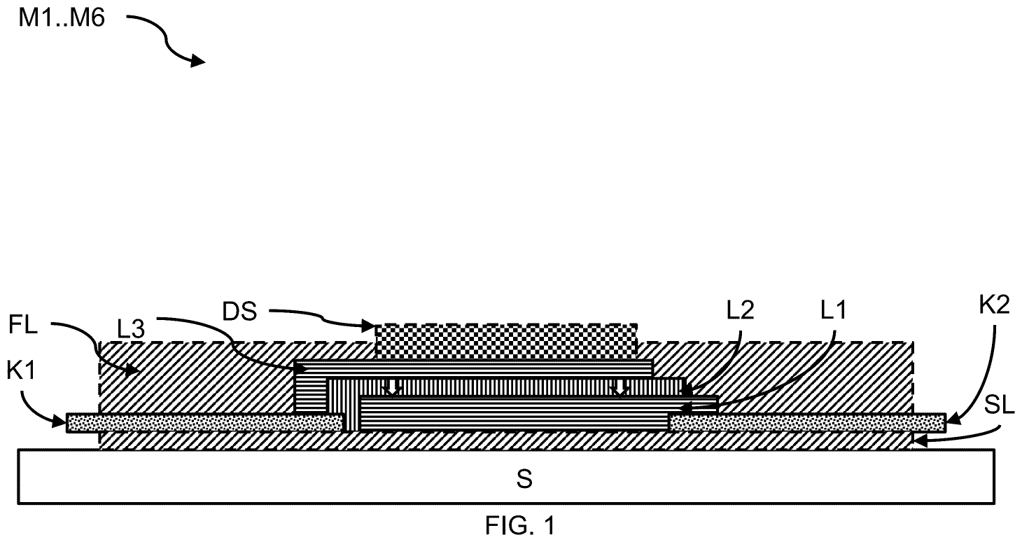

In particular, FIGS. 1 to 3 show parts of a device 1, wearable on the body, for the detection of a constriction of a lumen, particularly a stenosis in a blood vessel in a human or animal body.

The portable device 1 has a membrane-like substrate S which, upon use, is arranged on the outside of the lumen, particularly on the skin of the human or animal body.

Membrane-like means here that the substrate is thin compared to its extent. The material thickness can be reduced here to what is mechanically necessary, so that on the one hand a secure hold of the applied layers and structures is provided, but on the other hand also a certain mobility remains, so that the portable device 1 can shape itself to the outer contours of the skin at the site which is to be monitored, in order to enable a good transmission of sound or vibrations. In particular, the substrate has a thickness of less than 0.2 mm.

At least two sound converters M1, M2 are arranged on the membrane-like substrate S.

Each sound converter M1, M2 has, in turn, at least one stack of layers.

These layers are now explained further with reference to FIG. 1.

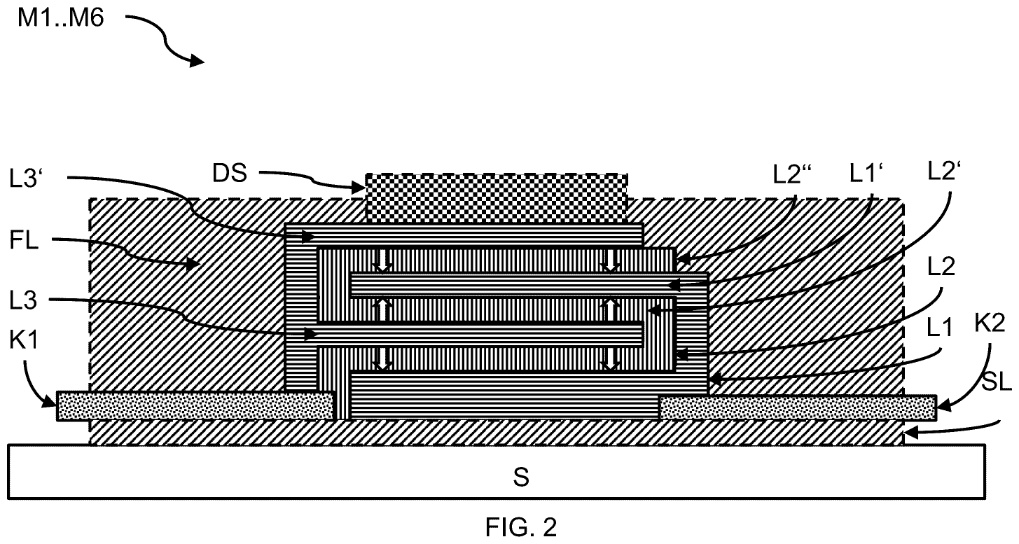

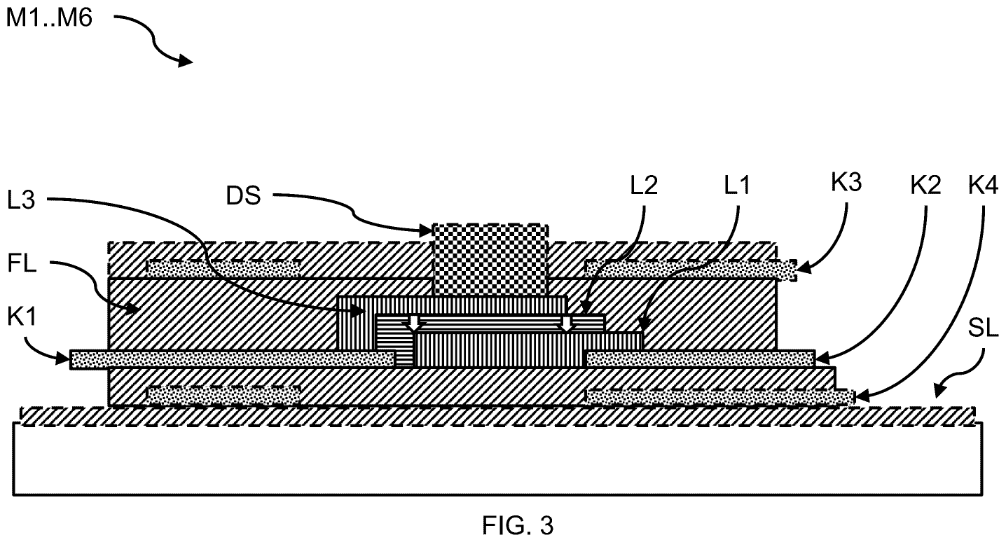

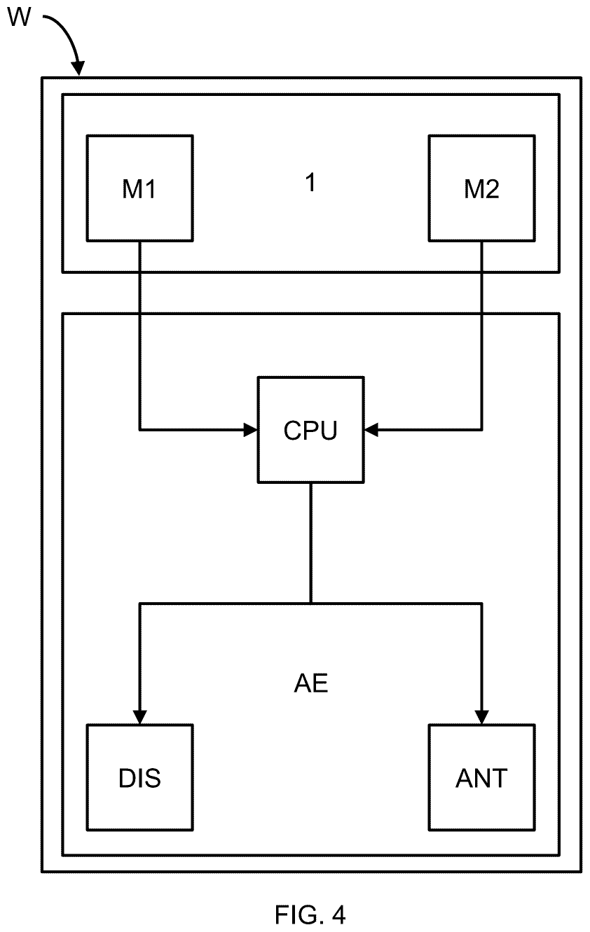

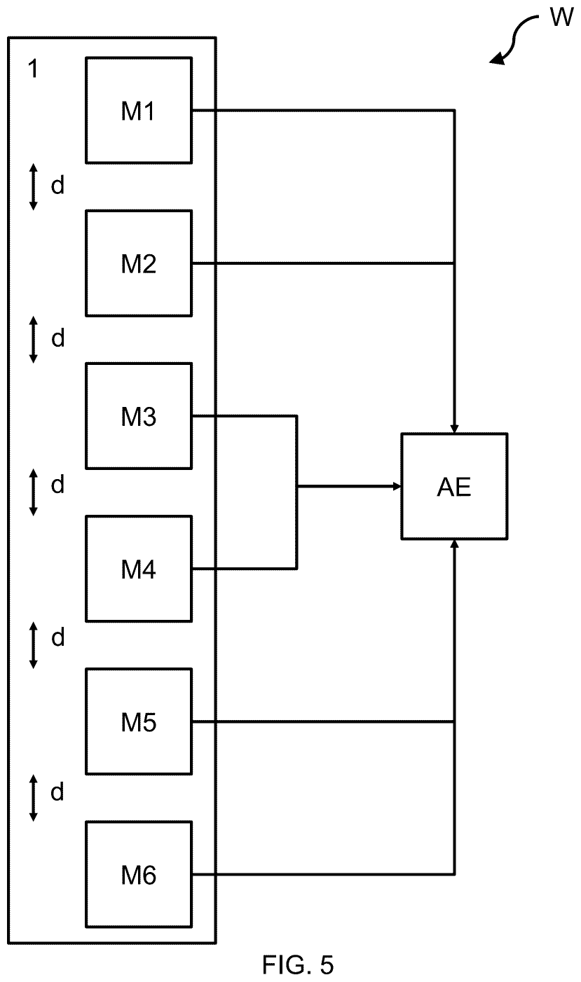

In so far as the layers have identical/similar characteristics, these are designated accordingly in FIG. 2 and FIG. 3. In addition, layers which are represented in the figures with hatching of similar type also have electrical characteristics of similar type. Generally, they then also comprise the same material, so that the number of different materials is small and hence also the processing effort and storage effort is small. Furthermore, in particular the layers which have a dashed frame in the figures are generally optionally.

The stack of layers has at least one partially covering sequence of a first electrically conductive layer L1, a PVDF layer L2 and a second electrically conductive layer L3. These constitute the actual core of the sound converters. As can be seen from FIGS. 1 to 3, the layers (in the sectional illustration) are arranged slightly offset with respect to one another, so that the layers L1 and L3 can be contacted at the edge.

In particular, provision can be made in embodiments of the invention that the first electrically conductive layer L1 and/or the second electrically conductor layer L3 comprise an electrically conductive plastic, in particular an electrically conductive polymer, and preferably PEDOT.PSS.

PEDOT.PSS is a polymer mixture of two ionomers and is also designated as poly (3,4-ethylenedioxythiophene) polystyrene sulphonate. PVDF designates polyvinylidene fluoride. PEDOT.PSS has, on the one hand, a high conductivity and, on the other hand, can be processed well. Therefore, PEDOT.PSS is particularly well suited as material for the electrically conducting layers.

Other electrically conductive polymers which can be used for the electrically conducting layers L1 and L3, are e.g. PANI (polyanaline), doped PPy (polypyrrole), and also doped PT (polythiophene).

The PVDF layer (L2) is electrically polarised perpendicularly to the layer structure and has a piezo effect.

On the side facing away from the substrate 5, furthermore a biocompatible cover layer DS is provided, wherein the first electrically conductive layer L1 (e.g. of PEDOT.PSS) and the second electrically conductive layer L3 (e.g. of PEDOT.PSS) are able to be connected to an evaluation unit AE.

Such a portable device 1 can be produced by means of screen printing, in which the individual layers are applied one after another. Other production methods are not ruled out, however. In particular, other printing methods, such as e.g. inkjet printers or other methods (e.g. by means of exposure methods) can be produced. It may be necessary here to reach appropriate temperatures during the production. For example, after applying, the PVFD layer L2 should be heated to a temperature above the Curie temperature ϑ Curie of approximately 145° C.

In the production, however, it is essential that the electrical polarisation of the PVDF layer(s) L2 can be brought about when the respective adjacent layers L1 and L3 are applied. For this process, the material must be exposed to an external voltage. The external voltage is dependent here on the material thickness. For a material thickness of 2 mm e.g. 150-300 V alternating voltage are used. The frequency of the alternating voltage can be selected here in a suitable manner, but is generally low, e.g. 1 Hz. Typically, the process does not take long, but rather can take place in the region of under a minute, in particular in less than half a minute. The polarising leads to the molecules being aligned into the desired b-phase, i.e. an anisotropic material with a high piezoelectric coefficient is achieved. The already processed contacts K1 and K2 to the layers L2 and L3 silver conductor tracks can be used as electrodes for this. All the sound converters M1 . . . M6 can be polarised simultaneously here. In FIGS. 1 to 3 the completed polarising of the layers is indicated by arrows. FIG. 2 shows by way of example a layer structure with several PVDF layers. Here, the electrical polarising of adjacent PVDF layers is (preferably) rotated through 180° with respect to one another. Thereby, it results particularly advantageously that the piezoelectric voltages of the PVDF layers can be added up particularly easily in the evaluation of the converters.

It is to be noted that this is possible not only with PVDF, but also with other polarisable materials.

Unlike in the prior art, however, here with the polarising the problem of short circuits does not arise, because when one of the contacting electrically conductive layers L1 (e.g. of PEDOT.PSS) and/or L3 (e.g. of PEDOT.PSS) short-circuits during the polarising, the corresponding section—unlike in the case of a section having a metallic component—is damaged irreversibly, so that the resistance there rises locally and the fault location does not appear. In addition, the electrically conductive layers L1 and/or L3 (e.g. of PEDOT.PSS) have a distinctly low roughness as e.g. layers containing silver, whereby the uniformity of a PVDF layer L2 is preserved even in the case of small layer thicknesses.

Such devices can be produced economically and permit a reliable and secure detection of (impending) stenoses.

Without loss of generality, provision can be made in embodiments of the invention that the membrane-like substrate S has a carrier membrane comprising polyethylene terephthalate (abbr. PET), polyurethane (abbr. PU), polyethylene naphthalate (abbr. PEN), polyimide (abbr. PI), polycarbonate (abbr. PC) or paper.

As the substrate side is not carried on the skin, this does not necessarily have to be biocompatible.

Thus on the basis of different material systems a device can be provided which can also be combined with different arrangements without this being ruled out through the choice of a particular material system.

In an embodiment of the invention at least two sound converters M1, M2 are pressure sensors or microphones or structure-borne sound converters.

This means that the invention permits diverse variation possibilities.

As shown in FIG. 2, provision can be additionally made that the stack of layers has several sequences of a first electrically conductive layer L1, L1′ (e.g. of PEDOT.PSS), a PVDF layer L2, L2′, L2″ and a second electrically conductive layer L3, L3′ (e.g. of PEDOT.PSS). Here, the stacks can be readable individually (not illustrated) or else can be interconnected internally.

With such an arrangement, it is possible to use on the same surface the effect of an acting (sound-/impulse wave/deflection) and to thus arrive at a better sensitivity. The piezo-electrically generated charge, and hence also the measured signal can be increased when electrically conductive layers (e.g. of PEDOT.PSS) and PVDF layers are arranged alternately one behind the other. Thereby, the effectively used sensor surface is multiplied per layer, therefore also the absolutely generated charge and hence the sensitivity of the sensor.

It is particularly advantageous when the second electrically conductive layer (e.g. of PEDOT.PSS) of a first sequence also forms at the same time the first electrically conductive layer (e.g. of PEDOT.PSS) of a second sequence.

Thus with simultaneous increase of the sensitivity, the stack size can be minimized.

According to a still further further development of the invention, the cover layer DS comprises polydimethylsiloxane (abbr. PDMS) or PET.

In particular, the cover layer can provide a mechanical impedance matching between the skin and an electrically conductive layer (e.g. of PEDOT.PSS) situated under the cover layer. Alternatively and/or additionally, the cover layer can also serve as contact protection and/or corrosion protection. Both PDMS and also the PET are biocompatible and can be in non-invasive contact with the skin.

According to a further further development of the invention, at least the stack of layers is embedded in a non-conducting material FL. In particular, the non-conducting material is PET or PU.

Hereby, a contact protection and/or corrosion protection and also an additional mechanical stability can also be provided for the layer structure.

In a still further further development of the invention, the connections K1, K2 of the first electrically conductive layer (e.g. of PEDOT.PSS) and/or of the second electrically conductive layer (e.g. of PEDOT.PSS) with the evaluation unit AE have a layer comprising silver.

Hereby, a secure, low-impedance connection can be provided.

According to another further further development of the invention, at least one further electrically conducting layer K3, K4 is arranged laterally adjacent—as shown in FIG. 3—to a stack.

By means of such further electrical layers K3, K4, a shield can be provided against interfering irradiations, in particular against mains hum. For this, the corresponding electrically conducting layer K3 and K4 can be connected to an earth-/ground potential of the evaluation unit AE.

In a still further further development of the invention, the electrically conducting layers K3 and K4 comprise silver.

Likewise for the adaptation between the substrate S and a first layer, e.g. for a contacting K1/K2, an adaptation layer SL can be provided.

The problem of the invention is furthermore solved by a system W for the detection of stenoses, having a portable device according to the invention and an evaluation unit AE, wherein the evaluation unit AE is connected to at least two sound converters M1, M2.

For example, the evaluation unit can be plugged by means of a plug connection on the portable device 1. The evaluation unit AE can also provide here a ground connection for shielding purposes.

The signals of each two sound converters M1, M2 are repeatedly evaluated differentially in operation, wherein on occurrence of a significant phase shift and/or a significant signal change at only one of the sound converters M1, M2 between a first evaluation and a second evaluation, chronologically following the first, a stenosis is detected.

The sampling rate can be selected here so that it can dissipate a typical range which is still perceptible by the human ear in a stethoscope. An example sampling frequency is 5 kHz.

The evaluation unit AE can also make available an (analog) filtering, in particular a low-pass filtering. Likewise, provision can be made that particular frequencies, e.g. mains hum (analog or digital) can be filtered out by means of a (notch) filter.

Likewise, the evaluation unit AE can also provide an amplification, e.g. by a transimpedance amplifier. This is generally connected to respectively a sound converter (by means of K1 and K2) using as small a connection line as possible.

In addition, for example, to a current supply (inductive/battery/energy harvesting), the evaluation unit AE can also comprise a corresponding processing unit CPU, e.g. a microcontroller or a suitably programmed switching circuit which activates a local error message arrangement DIS in the case of the detection of an (impending) stenosis and/or via a wireless interface ANT sends a signal to an external monitoring unit (not illustrated). For example, the evaluation unit AE can provide a wireless connection by means of Bluetooth/Bluetooth LowEnergy, ULE, or WLAN etc.

In particular, in a further development of the invention provision can be made that a significant phase shift has a phase shift of approximately 90°. The differential pressure change of two sound converters can be compared to one another. Particularly the change of the blood sounds (both high-frequency and also the low-frequency phase shift) before and after the stenosis is measurable and can be used in order to predict a bottleneck between two sensors.

Without loss of generality, provision can also be made—as shown in FIG. 5—that the portable device 1 has several sound converters M1 . . . M6, wherein the number can be selected in a suitable manner. Then respectively two sound converters can be read differentially.

The sound converters M1 . . . M6 preferably all have the same size and are preferably also situated at the same distance d, e.g. 10 . . . 15 mm, with respect to one another.

Although in the figures the sound converters M1 . . . M6 are illustrated as being square, this is not necessary. Rather, the sound converters could also be rectangular, elliptical or round. The size of the sound converters M1 . . . M6 should correspond here approximately to the dimension of the vascular system which is to be examined. For example, the sensor can have a diameter/edge length of 5 . . . 10 mm.

For example, the sound converters M1 and M2 can be read differentially. The sound converters M3 and M4 can be read simultaneously or subsequently. In turn, the sound converters M5 and M6 can be differentially read simultaneously or subsequently.

Alternatively, provision can also be made that the sound converters M1 and M4 are read differentially. The sound converters M2 and M5 can be read simultaneously or subsequently. In turn the sound converters M3 and M6 can be differentially read simultaneously or subsequently.

Such an arrangement with several sound converters offers the advantage that the lumen, i.e. a corresponding vascular access (shunt vein) can be monitored over a greater stretch, e.g. more than half of the forearm (or, if applicable, also upper arm). Thereby the occurrence of a constriction/stenosis can be monitored on a longer segment of a lumen, i.e. a longer venous segment.

The sound converters are preferably arranged in a line and thus reproduce approximately the course of the corresponding vascular access (shut vein).

According to the invention, an arrangement with at least two sound converters M1, M2 is used for detection. Thereby, a differential evaluation of the measurement signals is made possible, which greatly improves the sensitivity of the arrangement and, in addition, brings about more robust measurement results (i.e. fewer false-positive detections).

Such systems can be produced economically and permit a reliable and secure detection of (impending) stenoses. In particular here the portable device can be configured as a single-use article, whereas the evaluation unit can be re-used.

Without loss of generality, provision can also be made that the portable device 1 is arranged on an item of clothing or part of an item of clothing. The item of clothing could be e.g. a sleeve/arm sock, wherein the device described above is then preferably arranged on the inner side.

The evaluation unit AE can be provided in a permanent housing. Typically, the evaluation unit AE is configured so that it withstands mechanical stress and it is configured as a reusable product.

The portable device 1 can be usable once or several times. However, it is to be noted that with a heat disinfection, in certain circumstances a polarising must be carried out again.

The invention therefore makes it possible to monitor lumens of all kinds, particularly in a medical context.

Another example of monitoring a lumen—in addition to monitoring stenoses in blood vessels—is the detection of constrictions in a tube, e.g. for extracorporeal blood treatment during treatment.

For example, the tube could be constricted by kinking a tube or by pressure (pinching, patient sitting on the tube).

The device according to the invention can also be used here, in which the corresponding lumen “wears” the portable device 1, e.g. by the portable device 1 is placed around a tube/lumen. The portable device 1 or its evaluation unit AE can be connected to a blood treatment machine, and the portable device 1 can also be part of a blood treatment machine or a separate device that, for example, sends data about a detected constriction/stenosis as a (digital) signal. Such a (digital) signal can, for example, be transmitted to an external server, a wearable device such as a smartphone or smartwatch, a treatment machine, etc.

### LIST OF REFERENCE NUMBERS

1 device wearable on the body
S membrane-like substrate
M1 . . . 6 sound converter
L1,L3;L1',L3′ electrically conducting layer (e.g. of PEDOT.PSS)
L2,L2′,L2″ PVDF layer
DS cover layer
AE evaluation layer
FL non-conducting material
K1, K2 connection
K3, K4 further electrically conducting layer
CPU processing unit
ANT wireless interface
d distance

1. A portable device for the detection of a constriction in a lumen, wherein the portable device has a membrane-like substrate which, in use, is arranged on the outside of the lumen, wherein at least two sound converters are arranged on the membrane-like substrate, wherein each sound converter has at least one stack of layers, wherein the stack of layers comprises at least one partially covering sequence of a first electrically conductive layer, a PVDF layer and a second electrically conductive layer, wherein the PVDF layer is polarised electrically perpendicularly to the layer structure and has a piezo effect, wherein on the side facing away from the substrate furthermore a biocompatible cover layer is provided, wherein the first electrically conductive layer and the second electrically conductive layer are able to be connected to an evaluation unit.

2. The device according to claim 1, wherein the membrane-like substrate has a carrier membrane comprising PET, PU, PEN, PI or PC.

3. The device according to claim 1, wherein the at least two sound converters are pressure sensors or microphones or structure-borne sound converters.

4. The device according to claim 1, wherein the stack of layers comprises several sequences of a first electrically conductive layer, a PVDF layer and a second electrically conductive layer.

5. The device according to claim 1, wherein the cover layer comprises PDMS or PET.

6. The device according to claim 1, wherein the cover layer provides a mechanical impedance matching between the skin and an electrically conductive layer situated under the cover layer.

7. The device according to claim 1, wherein the stack of layers comprises several sequences of a first electrically conductive layer, a PVDF layer and a second electrically conductive layer, wherein the second electrically conductive layer of a first sequence at the same time also forms the first electrically conductive layer of a second sequence.

8. The device according to claim 1, wherein at least the stack of layers is embedded in a non-conducting material.

9. The device according to claim 8, wherein the non-conducting material is PET or PU.

10. The device according to claim 1, wherein the connections of the first electrically conductive layer and/or of the second electrically conductive layer with the evaluation unit have a layer comprising silver.

11. The device according to claim 1, wherein at least one further electrically conducting layer is arranged laterally adjacent to a stack.

12. The device according to claim 1, wherein the first electrically conductive layer and the second electrically conductive layer comprises PEDOT.PSS.

13. The device according to claim 1, wherein the portable device is designed for wearing on a human or animal body, wherein upon use the portable device is arranged on the skin of a human or animal body for detecting a stenosis in a blood vessel of the human or animal body.

14. A system for the detection of constrictions, having the device according to claim 1 and an evaluation unit, wherein the evaluation unit is connected to the at least two sound converters, wherein the signals of each two sound converters are repeatedly evaluated differentially, wherein on occurrence of a significant phase shift and/or of a significant signal change at only one sound converter between one evaluation and a second evaluation, chronologically following the first, a stenosis is detected.

15. The system according to claim 14, wherein a significant phase shift comprises a phase shift of approximately 90°.

16. A portable device that is wearable on the body for the detection of a constriction in a lumen, wherein the portable device has a membrane-like substrate which, in use, is arranged on the outside of the lumen, wherein at least two sound converters are arranged on the membrane-like substrate, wherein each sound converter has at least one stack of layers, wherein the stack of layers comprises at least one partially covering sequence of a first electrically conductive layer, a PVDF layer and a second electrically conductive layer, wherein the PVDF layer is polarised electrically perpendicularly to the layer structure and has a piezo effect, wherein on the side facing away from the substrate furthermore a biocompatible cover layer is provided, wherein the first electrically conductive layer and the second electrically conductive layer are able to be connected to an evaluation unit.

17. The device according to claim 2, wherein the portable device is wearable on the body.

18. The device according to claim 3, wherein the portable device is wearable on the body.

19. The device according to claim 4, wherein the portable device is wearable on the body.

20. The device according to claim 5, wherein the portable device is wearable on the body.
